(19) United States
(12) Patent Application Publication
(10) Pub. No.: US 2007/0299457 A1
(43) Pub. Date: Dec. 27, 2007

(54) Ear cleaning device

(76) Inventor: Diamantina Morales, Mission, TX (US)

Correspondence Address: LITMAN LAW OFFICES, LTD., P.O. BOX 15035, CRYSTAL CITY STATION, ARLINGTON, VA, 22215, US

(21) Appl. No.: 11/812,592

(22) Filed: Jun. 20, 2007

Related U.S. Application Data
(60) Provisional application No. 60/816,352, filed on Jun. 26, 2006.

(51) Int. Cl. A61F 11/00

(52) U.S. Cl. 606/162; 604/1

(57) ABSTRACT

The ear cleaning device is a device for removing wax from an ear. The ear cleaning device includes a shaft having opposed first and second ends. First and second cleaning heads are respectively mounted to the first and second ends of the shaft. The first cleaning head is formed from an absorbent material. The second cleaning head is formed from a loop coated with the absorbent material. The loop may be used to abrade and engage the wax, secretions, bodily waste and debris within the ear, with the absorbent material aiding in adhering the waste to the device for removal thereof. The first cleaning head may further be used for subsequently gently cleaning the ear and for absorbing fluids that may be trapped therein.

### CROSS-REFERENCE TO RELATED APPLICATION

This application claims the benefit of U.S. Provisional Patent Application Ser. No. 60/816,352, filed Jun. 26, 2006.

### BACKGROUND OF THE INVENTION

1. Field of the Invention

The present invention relates to personal hygiene devices, and particularly to an ear cleaning device for removing wax from an ear.

2. Description of the Related Art

Over a period of time, the body builds up wax in the ears. As a stopgap measure, people often insert a finger into the ear and use a fingernail to scrape the wax from the ear. However, this method of cleaning the ear is often ineffective because the finger often cannot be inserted far enough into the inner ear canal, and is also considered unhygienic because the wax becomes lodged under the fingernails, where it is unsightly and may support the growth of microorganisms.

Alternatively, some people insert the hooked end of a bobby pin or the tip of a pencil or other sharp, narrow diameter object into the ear to break up hard, encrusted wax buildups. This practice is often considered dangerous, as such rigid or sharp objects may accidentally puncture the ear drum. Cotton swabs are generally found to be both safer and more effective, but the balls of cotton formed at the opposite ends of the swabs are often not effective at breaking up the wax when the wax has hardened. Thus, an ear cleaning device solving the aforementioned problems is desired.

### SUMMARY OF THE INVENTION

The ear cleaning device is a device for cleaning wax from an ear. The ear cleaning device may be formed from disposable materials and is portable. The ear cleaning device has a shaft having opposed first and second ends. The shaft may be formed from wood, hard plastic, cardboard or the like, and preferably has a substantially elongated, cylindrical contour. The shaft is preferably slightly flexible.

First and second cleaning heads are respectively mounted to the first and second ends of the shaft. The first cleaning head is formed from an absorbent material, and preferably has a substantially bulbous contour. The second cleaning head is formed from a loop coated with the absorbent material. Preferably, the loop is substantially U-shaped, the arms of the loop being joined to the shaft so that the bight of the loop projects from the end of the device.

The loop may be used to abrade and engage hard encrustations of wax in the ear, with the absorbent material aiding in adhering the wax to the device for removal thereof and preventing the tissues of the inner ear and ear drum from being debrided by the loop. The first cleaning head may further be used for subsequently gently cleaning the ear and for absorbing fluids that may be trapped therein. It should be understood that the ear cleaning device may be used for any desired purpose and is not only provided for the cleaning of a user's ears; the ear cleaning device is multi-functional, similar to the variety of functions for which one would use a conventional cotton swab, for example.

These and other features of the present invention will become readily apparent upon further review of the following specification and drawings.

### BRIEF DESCRIPTION OF THE DRAWINGS

FIG. 1 is an environmental, perspective view of an ear cleaning device according to the present invention, showing the loop end being used to clean the external ear.

FIG. 2 is a front view of the ear cleaning device according to the present invention.

FIG. 3 is an environmental, perspective view of the ear cleaning device according to the present invention in use, showing the loop end of the device disposed within the inner ear before scraping the wax.

FIG. 4 is an environmental, perspective view of the ear cleaning device according to the present invention in use similar to FIG. 3, but showing the wax adhering to the loop end of the device after cleaning the inner ear.

Similar reference characters denote corresponding features consistently throughout the attached drawings.

### DETAILED DESCRIPTION OF THE PREFERRED EMBODIMENT

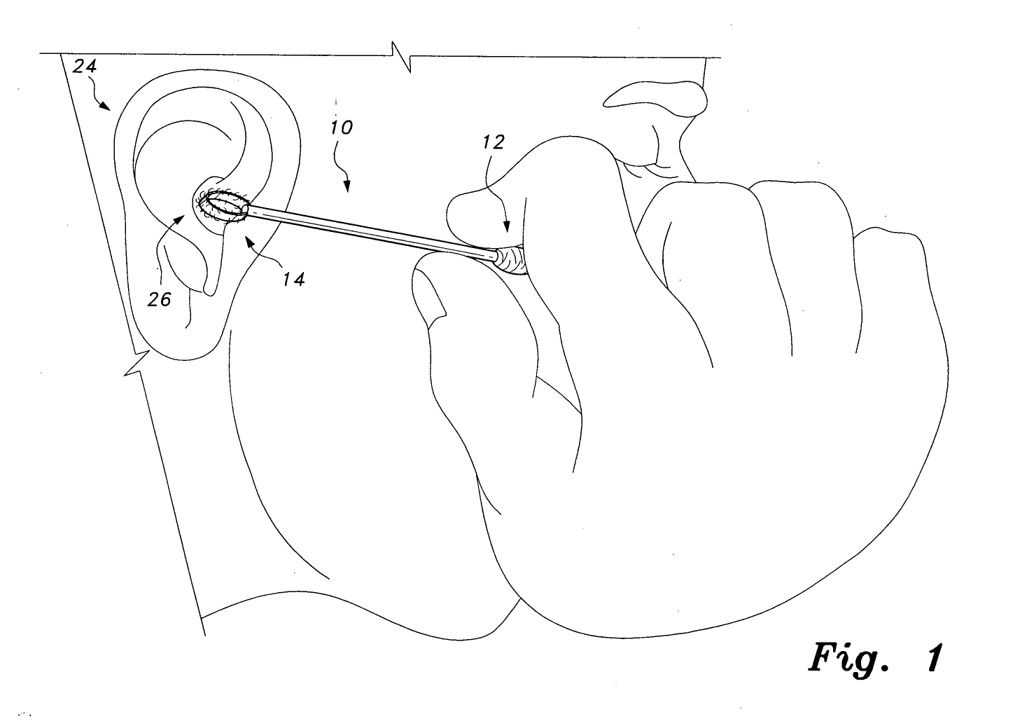

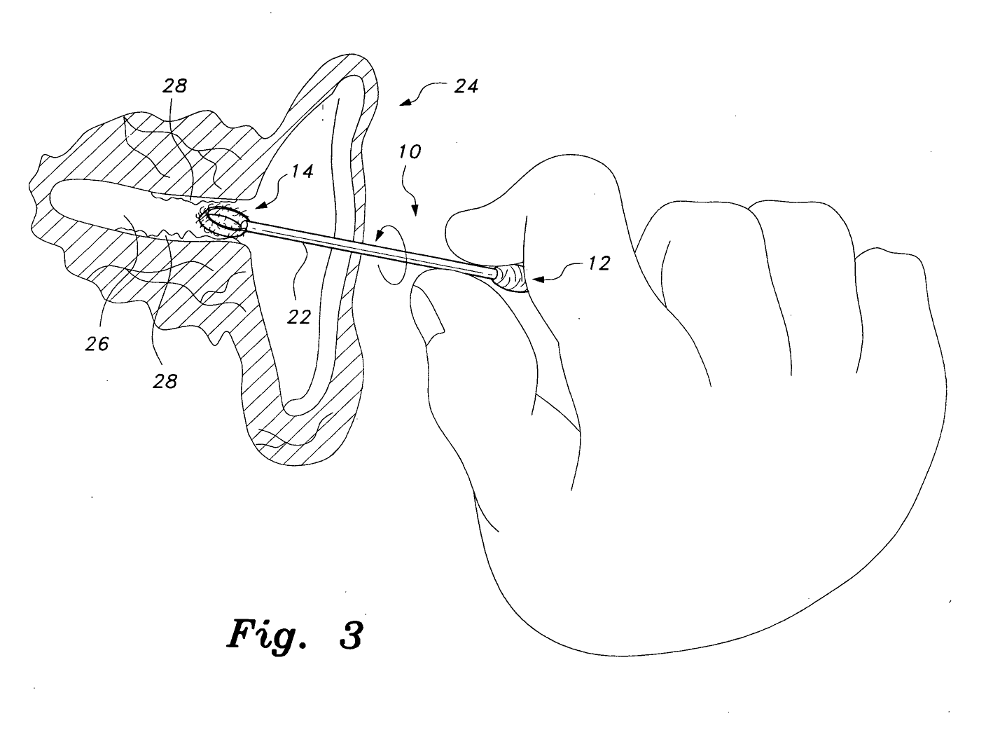

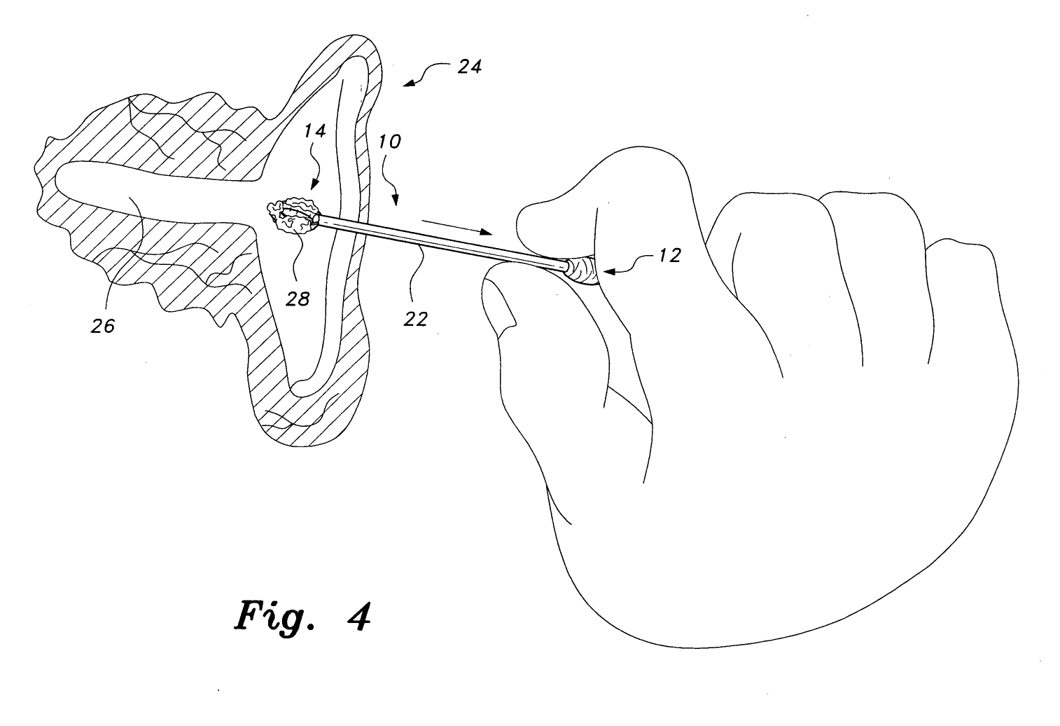

The present invention is directed towards an ear cleaning device 10, as shown in FIGS. 1-4. The ear cleaning device 10 is a personal hygiene device for removing wax from an ear, such as the removal of ear wax 28 from the ear canal 26 of a user's ear 24, as shown in FIGS. 3 and 4. It should be understood that, as in the case of a cotton swab, although the primary use of the ear cleaning device is for cleaning the ear, the ear cleaning device 10 may be used for the removal of any bodily secretions, waste or debris, such as mucus, from any orifice of the body that can receive the tips of the device. The ear cleaning device 10 may be formed from disposable materials and is portable. It should be understood that the ear cleaning device may be used for any desired purpose and is not only provided for the cleaning of a user's ears; the ear cleaning device is multi-functional, similar to the variety of functions for which one would use a conventional cotton swab, for example.

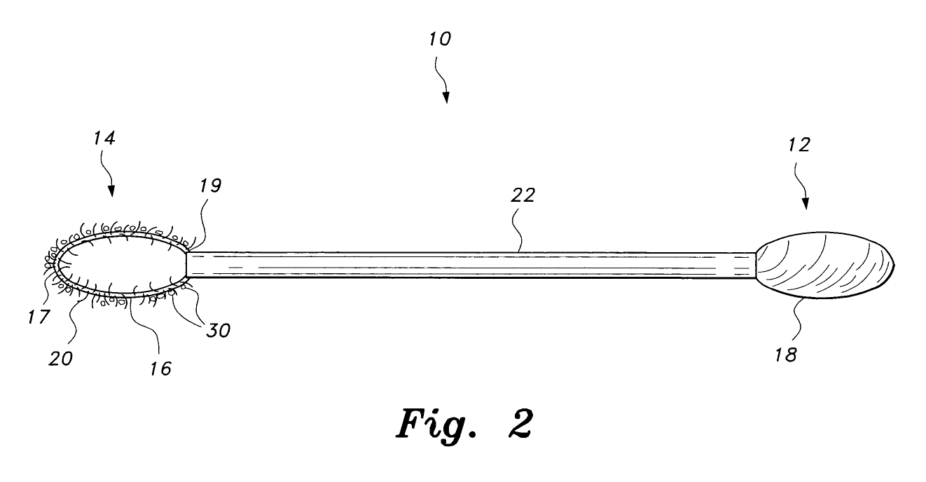

As best shown in FIG. 2, the ear cleaning device 10 includes a shaft 22 having opposed first and second ends. The shaft 22 may be formed from wood, hard plastic, cardboard or the like. Shaft 22 is adapted to be grasped by the user (as illustrated in FIG. 1) and, in the preferred embodiment, shaft 22 is approximately three inches in length. The shaft 22 preferably has a substantially cylindrical contour, and is preferably slightly flexible, so that the tips will bend before accidentally puncturing tissues, and so that the shaft 22 can better negotiate channels, such as the ear canal.

First and second cleaning heads 12, 14 are respectively mounted to the first and second ends of the shaft 22. The first cleaning head 12 is formed from an absorbent material 18, and preferably has a substantially bulbous contour. Absorbent material 18 may be cotton, foam, sponge, or any other suitable material for the absorption of fluids and the gentle cleaning of bodily orifices. First cleaning head 12 may be used, for example, for the removal of water trapped within ear canal 26.

The second cleaning head 14 is formed from a loop 16 coated with absorbent material 20. The core of loop 16 may be formed from a rigid material, or may be formed from a material having sufficient flexibility and resilience that the loop will bend when too much pressure is applied, but regain its shape when the pressure is removed, so that the loop will not puncture or debride sensitive tissues. Preferably, absorbent material 20 is formed from the same material as absorbent material 18.

In the preferred embodiment, the loop 16 is formed from a substantially U-shaped frame, having a bight 17 and a pair of arms 19 joined to the shaft 22. Loop 16 is adapted to be received within a bodily orifice, such as ear canal 26, and is appropriately dimensioned and configured.

The loop 16 may be used to abrade and engage wax buildups within the ear, with the absorbent material 20 aiding in adhering the waste to the device for removal thereof. The first cleaning head 12 may further be used for subsequently gently cleaning the ear and for absorbing fluids that may be trapped therein. The loop 16 of second cleaning head 14 may be formed from metal, plastic or any other suitable material. Further, absorbent material 20 may have dry soap or particles 30 of other cleaning products infused or embedded therein, or otherwise formed thereon, so as to aid in the cleaning of the bodily orifice. Water or other solvent may be added to the absorbent material 20 (and the dry soap 30 infused therein) prior to insertion into the bodily orifice, or, for example, water trapped in the ear canal may be used to dissolve and activate the cleaning material 30. Cleaning material 30 may be any suitable water-soluble cleanser.

As shown in FIG. 3, in use, the second cleaning head 14 is inserted into the ear canal 26 for the removal of ear wax 28 therefrom. Rotation of the shaft (indicated by the directional arrow) allows the loop 16 to abrade the ear wax 28 from the walls of ear canal 26. The absorbent material 20, which coats loop 16, aids in adhering the ear wax 28 to the loop 16 (as shown in FIG. 4) and further absorbs fluids which may be trapped within the ear canal 26, or which form part of the secretions or bodily waste being removed.

As shown in FIG. 4, once the loop 16 has abraded the ear wax 28, the ear wax 28 adheres to the material 20, and is held onto and within the loop 16. The user then removes the second head 14 from the ear canal (as indicated by the directional arrow of FIG. 4) for disposal of the device. The first cleaning head 12 may be subsequently inserted into the ear canal 26 for removal of smaller debris and fluids, and for subsequent gentle cleaning of the ear canal 26. As noted above, the cleaning of ear canal 26 is shown and described for exemplary purposes only, and device 10 may be used in the cleaning of any suitable bodily orifice, or for cleaning external portions of the user's body. It should be further noted that when in use in the cleaning of ear canal 26 or other sensitive bodily areas, which may be subject to damage or injury, the user should apply gentle pressure to device 10 during cleaning and use caution so that sensitive and delicate body parts are not accidentally punctured or abraded.

It is to be understood that the present invention is not limited to the embodiment described above, but encompasses any and all embodiments within the scope of the following claims.

I claim:

1. An ear cleaning device, comprising:
a shaft having opposed first and second ends;
a first cleaning head formed of an absorbent material, the first cleaning head being mounted on the first end of the shaft; and
a second cleaning head mounted on the second end of the shaft, the second cleaning head being a loop coated with the absorbent material, the first and second cleaning heads being adapted for insertion into an ear for removal of bodily waste and debris therefrom.

2. The ear cleaning device as recited in claim 1, wherein the loop has a substantially U-shaped contour, the loop having a pair of arms attached to the shaft and a bight projecting from the second end of said shaft.

3. The ear cleaning device as recited in claim 1, wherein the loop is formed from a substantially rigid material.

4. The ear cleaning device as recited in claim 1, wherein the loop is formed from a resilient material.

5. The ear cleaning device as recited in claim 1, wherein the loop is formed from a material selected from the group consisting of: metal, plastic and combinations thereof.

6. The ear cleaning device as recited in claim 1, wherein the shaft is formed from a substantially rigid material.

7. The ear cleaning device as recited in claim 1, wherein the shaft is formed from a resilient material.

8. The ear cleaning device as recited in claim 1, wherein the shaft is formed from a material selected from the group consisting of: wood, plastic, cardboard and combinations thereof.

9. The ear cleaning device as recited in claim 1, wherein the first cleaning head has a substantially bulb-shaped contour.

10. The ear cleaning device as recited in claim 1, wherein the absorbent material is a water-absorbent material.

11. The ear cleaning device as recited in claim 10, wherein the absorbent material is selected from the group consisting of: cotton, foam, sponge and combinations thereof.

12. The ear cleaning device as recited in claim 10, further comprising a water soluble cleanser embedded within the absorbent material.

13. The ear cleaning device as recited in claim 12, wherein the water soluble cleanser is soap.
